SEQUENCE LISTING (14 sequences)

SEQ ID NO: 1 - DNA, 3495 bp, Homo sapiens
ggggcagtattctgtgttgagggaggaaaaacactcccttccaaaagcatgacaggcagaaagcagagaagggccaggactggctgagggcggggagctgggcctctggggtggacacacccttggtcacattgtgagggtagcttggttggccagtcccaccactgcagtgaccacagttgtgttgggctcacaccagtgaaccgaagctctggattctgagagtctgaggattccgtgaagatctcagacttgggctcagagcaaggatgcgtgaaattgtccatattcagattggccagtgtggcaaccagatcggagccaagttctgggagatgattggtgaggaacacgggatcgacttggctgggagcgaccgcggggcctcggccttgcagctggagagaatcagcgtgtactacaacgaagcctacggtaggaaatatgtgccccgagcagtcttggtggacctagaacctgggacgatggacagcattcgatctagcaaattaggagctctctttcaacccgacagttttgtccatggtaactctggggctggcaacaactgggccaaaggccactacacggagggagccgagctgatcgagaatgtcctagaggtggtgaggcacgagagtgagagctgtgactgcctgcagggcttccagatcgtccactccctgggcgggggcacaggctccgggatgggcactctgctcatgaacaagattagagaggagtacccggaccggatcatgaattccttcagcgtcatgccttctcccaaggtgtcggacacggtggtggagccctacaacgcggttctgtctatccaccagctgattgagaatgcagatgcctgtttctgcattgacaatgaggccctctatgacatctgcttccgtaccctgaagctgacgacacccacctatggggatctcaaccacctagtgtccttgaccatgagcggcataaccacctccctccggttcccgggtcagctcaacgcagacctgcgcaagctggcggtgaacatggtccccttcccccgcctgcacttctttatgcccggctttgccccactcacggcccagggcagccagcagtaccgagccctctccgtggccgagctcacccagcagatgttcgatgcccgcaataccatggctgcctgtgacctccgccgtggccgctacctcacagtggcctgcattttccggggcaagatgtccaccaaggaagtggaccagcaactgctctccgtgcagaccaggaacagcagctgctttgtggagtggattcccaacaacgtcaaggtggctgtctgcgacatcccgccccgggggctgagcatggccgccaccttcattggcaacaacacggccatccaagagatctttaatagggtctctgagcatttctcagccatgttcaaaaggaaagcttttgtgcactggtacaccagcgaagggatggacataaacgaatttggggaagctgaaaataacatccatgatttggtatccgagtaccaacaatttcaagatgccaaagcagttctagaggaagatgaagaggtcacggaggaggcagaaatggagccagaagataagggacattaactgtgagagaagctgtgccgcggagtcgcttacagaacagtttctcattagatgagtgtttctcctgcagcactccaaaacccactctgcactgcagcacagtgaatgatatgcactcaccattagcttcgacacagggactgagggagacaggtggggagcagctgacaggcattagggtctttgctgacatctactaaccttgaagagtttgatgttcagtgcatacttattaacttaaaaaaatagcaaatttattgtaaagtgctccctttgtttcaaagtgtttgccaggcatccagactacacgtgtggatttgcagggagccactggagttggtgttacatttttatactttagcagcactgataggcaccctggaatcctcacttggtatccgagggctactaagactctttccttaggttctttcctctgagcaaacactgactggcatcctgctttccagtgcctgccagcctccagaagagccaggtgcctgactagtacatggggagctacagagccaaggtcaatgtgagtcaacatccactagaaatatccatgttgtgtagacctgtgcatacaacatgctaactggaaaagaggaaaaaagaaaagccacagtcctctccacaaaaatacctggtccaaacaagaaaaacaaaaagacaagcaaaactaaagaactgcagtcttctgatctttatttctgaagagctagcctttaacatatatgtttatatagtttaaatttcttactactgttagatcccaggaattcattaataatcatccttggctttccttttaaaggctattttgaaatggtcttttcactttcattcagtcatcaccccccaaaatgctctgcagcctctctgctctttgagaaagggcacaccatgcgctcggcaaccattcaaatgcaggaattaagcagcaatggctgcagtgtccttctcagttatggaggacatcgtctcattagggaacttttacagttcaaattaatttgcagaagttgccataaatgtttgcataatgacatagctttagcactacatgattttaatctgctcacattataacaggaccaaatacacaagagcgtaatcaaatcatctgtaacttcttaattacagtttacctatttctgacatgcagcactgccatctcttccagcaccatcagggttttaatggccctctagaattaccactgagatacactatttgatccatggataaccggtaatgggaaaatgctccgaccctcaatgcagtaaatatttacttgcaggcaactgggttctcatctcttgatttgcttttgtaatcagcaataataaaatagcaggtagatggatgacagttgctcattctgagaaacttcactcttttcacttatgcatcacgaggaaataactaaaatacataccaagagaaaaataccttgccatcggatcatcaacaagtcttctatttacaaacttcaaaaaacacaaaacaacattcatgttttaaatgctttctacttgtggttcaagaagcactagatttagtaagaaactctacctatatacttagtttgaagttagtaacttcctgagatgctaaagacttacagcctgcgattatacaaggatttacacatgcttcctctggtgctttacttcccaaacctaaaaaagcaatgaaatagatgtaaggaaggagggatttaaaccttttaaaaaacttttgctgacttatattactgtaaagatttgtttgctcaatagtaatcattaaactacaaagtaattcaattttaaatggcaaaattgctttatttcagactaaataaattccttttcttgaagcctaa

SEQ ID NO: 2 - DNA, 1356 bp, Homo sapiens
atgcgtgaaattgtccatattcagattggccagtgtggcaaccagatcggagccaagttctgggagatgattggtgaggaacacgggatcgacttggctgggagcgaccgcggggcctcggccttgcagctggagagaatcagcgtgtactacaacgaagcctacggtaggaaatatgtgccccgagcagtcttggtggacctagaacctgggacgatggacagcattcgatctagcaaattaggagctctctttcaacccgacagttttgtccatggtaactctggggctggcaacaactgggccaaaggccactacacggagggagccgagctgatcgagaatgtcctagaggtggtgaggcacgagagtgagagctgtgactgcctgcagggcttccagatcgtccactccctgggcgggggcacaggctccgggatgggcactctgctcatgaacaagattagagaggagtacccggaccggatcatgaattccttcagcgtcatgccttctcccaaggtgtcggacacggtggtggagccctacaacgcggttctgtctatccaccagctgattgagaatgcagatgcctgtttctgcattgacaatgaggccctctatgacatctgcttccgtaccctgaagctgacgacacccacctatggggatctcaaccacctagtgtccttgaccatgagcggcataaccacctccctccggttcccgggtcagctcaacgcagacctgcgcaagctggcggtgaacatggtccccttcccccgcctgcacttctttatgcccggctttgccccactcacggcccagggcagccagcagtaccgagccctctccgtggccgagctcacccagcagatgttcgatgcccgcaataccatggctgcctgtgacctccgccgtggccgctacctcacagtggcctgcattttccggggcaagatgtccaccaaggaagtggaccagcaactgctctccgtgcagaccaggaacagcagctgctttgtggagtggattcccaacaacgtcaaggtggctgtctgcgacatcccgccccgggggctgagcatggccgccaccttcattggcaacaacacggccatccaagagatctttaatagggtctctgagcatttctcagccatgttcaaaaggaaagcttttgtgcactggtacaccagcgaagggatggacataaacgaatttggggaagctgaaaataacatccatgatttggtatccgagtaccaacaatttcaagatgccaaagcagttctagaggaagatgaagaggtcacggaggaggcagaaatggagccagaagataagggacattaa

SEQ ID NO: 3 - protein, 451 aa, Homo sapiens
MREIVHIQIGQCGNQIGAKFWEMIGEEHGIDLAGSDRGASALQLERISVYYNEAYGRKYVPRAVLVDLEPGTMDSIRSSKLGALFQPDSFVHGNSGAGNNWAKGHYTEGAELIENVLEVVRHESESCDCLQGFQIVHSLGGGTGSGMGTLLMNKIREEYPDRIMNSFSVMPSPKVSDTVVEPYNAVLSIHQLIENADACFCIDNEALYDICFRTLKLTTPTYGDLNHLVSLTMSGITTSLRFPGQLNADLRKLAVNMVPFPRLHFFMPGFAPLTAQGSQQYRALSVAELTQQMFDARNTMAACDLRRGRYLTVACIFRGKMSTKEVDQQLLSVQTRNSSCFVEWIPNNVKVAVCDIPPRGLSMAATFIGNNTAIQEIFNRVSEHFSAMFKRKAFVHWYTSEGMDINEFGEAENNIHDLVSEYQQFQDAKAVLEEDEEVTEEAEMEPEDKGH

SEQ ID NO: 4 - protein, 21 aa, Artificial Sequence
  Description of Artificial SequencePrimer
VLEEDEEVTEEAEMEPEDKGH

SEQ ID NO: 5 - DNA, 19 bp, Artificial Sequence
  Description of Artificial SequencePrimer
tgactggcatcctgctttc

SEQ ID NO: 6 - DNA, 19 bp, Artificial Sequence
  Description of Artificial SequencePrimer
gcataactggactgatttc

SEQ ID NO: 7 - DNA, 18 bp, Artificial Sequence
  Description of Artificial SequencePrimer
tgtaaaacgacggccagt

SEQ ID NO: 8 - DNA, 18 bp, Artificial Sequence
  Description of Artificial SequencePrimer
caggaaacagctatgacc

SEQ ID NO: 9 - DNA, 20 bp, Artificial Sequence
  Description of Artificial SequencePrimer
aattaaccctcactaaaggg

SEQ ID NO: 10 - DNA, 20 bp, Artificial Sequence
  Description of Artificial SequencePrimer
taatacgactcactataggg

SEQ ID NO: 11 - DNA, 23 bp, Artificial Sequence
  Description of Artificial SequencePrimer
actcactatagggctcgagcggc

SEQ ID NO: 12 - DNA, 22 bp, Artificial Sequence
  Description of Artificial SequencePrimer
cacggaatcctcagactctcag

SEQ ID NO: 13 - DNA, 26 bp, Artificial Sequence
  Description of Artificial SequencePrimer
gttgtagtaccacgctgattctctcc

SEQ ID NO: 14 - DNA, 63 bp, Artificial Sequence
  Description of Artificial SequencePrimer
gttctagaggaagatgaagaggtcacggaggaggcagaaatggagccagaagataagggacat

(19) United States
(12) Patent Application Publication
(10) Pub. No.: US 2004/0121351 A1
(43) Pub. Date: Jun. 24, 2004

(54) Nucleic acid sequence for a human beta tubulin

(76) Inventors: Ingo Hansmann, Halle/Saale (DE); Kietmar Scholte, Gottingen (DE); Claudia Gross, Regensburg (DE)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/415,420

(22) PCT Filed: Oct. 31, 2001
(86) PCT No.: PCT/EP01/12631

(51) Int. Cl. C12Q 1/68; C07K 14/47; C07H 21/04

(52) U.S. Cl. 435/6; 435/69.1; 435/320.1; 435/325; 530/358; 536/23.5

(57) ABSTRACT

The present invention is related to a nucleic acid molecule comprising a nucleotide sequence encoding the C terminal end of a tubulin molecule, wherein the C terminal end comprises the following sequence:

VLEEDEEVTEEAEMEPEDKGH (SEQ ID. No. 4)

The present invention is related to nucleic acid molecules comprising a nucleotide sequence encoding a tubulin molecule, vectors containing said nucleic acids, cells comprising such vectors, an amino acid sequence coded by such nucleic acid molecule, use of such nucleic acids and methods for production of such tubulin molecules, for diagnosing polymorphism in a tubulin molecule and for the screening of anticancer drugs.

Microtubules are essential to the eucaryotic cell due as they are involved in many processes and functions such as, e.g., being components of the cytoskeleton, of the centrioles and ciliums and in the formation of spindle fibres during mitosis. The constituents of microtubules are heterodimers consisting of one α-tubulin molecule and one β-tubulin molecule. These two related self-associating 50 kDa proteins are encoded by a multigen family. The various members of this multigen family are dispersed all over the human genome. Both α-tubulin and β-tubulin are most likely to originate from a common ancestor as their amino acid sequence shows a homology of up to 50%. In man there are at least 15 genes or pseudogenes for β-tubulin.

The conservation of structure and regulatory functions among the β-tubulin genes in three vertebrate species (chicken, mouse and human) allowed the identification of and categorization into six major classes of beta-tubulin polypeptide isotypes on the basis of their variable carboxyterminal ends. The specific, highly variable 15 carboxyterminal amino acids are very conserved among the various species. Beta-tubulins of categories I, II, and IV are closely related differing only 2-4% in contrast to categories III, V and VI which differ in 8-16% of amino acid positions [Sullivan K. F., 1988, Ann. Rev. Cell Biol. 4: 687-716].

Also the expression pattern is very similar between the various species as can be taken from the following table [Sullivan K. F., 1988, Ann. Rev. Cell Biol. 4: 687-716] which comprises the respective human members of each class:

| isotype | member | expression pattern |
|---|---|---|
| class I | HM 40 | ubiquitous |
| class II | H β 9 | mostly in the brain |
| class III | H β 4 | exclusively in the brain |
| class IVa | H β 5 | exclusively in the brain |
| class IVb | H β 2 | ubiquitous |

The C terminal end of the beta-tubulins starting from amino acid 430 is regarded as highly variable between the various classes. Additionally, the members of the same class seem to be very conserved between the various species.

As tubulin molecules are involved in many processes and form part of many structures in the eucaryotic cell, they are possible targets for pharmaceutically active compounds. As tubulin is more particularly the main structural component of the microtubules it may act as point of attack for anticancer drugs such as vinblastin, colchicin, estramustin and taxol which interfere with microtubule function. The mode of action is such that cytostatic agents such as the ones mentioned above, bind to the carboxyterminal end the β-tubulin which upon such binding undergoes a conformational change. For example, Kavallaris et al. [Kavallaris et al. 1997, J. Clin. Invest. 100: 1282-1293] reported a change in the expression of of specific β-tubulin isotypes (class I, II, III, and IVa) in taxol resistant epithelial ovarian tumor. It was concluded that these tubulins are involved in the formation of the taxol resistence. Also a high expression of class III β-tubulins was found in some forms of lung cancer suggesting that this isotype may be used as a diagnostic marker.

The problem underlying the present invention was to provide the means to further characterize the various tubulins present in eucaryotic cells. A further problem underlying the present invention was to provide the means to extend possible screening programs for cytostatic agents to other isotypes of human beta-tubulins.

This problem is solved in a first aspect by a nucleic acid molecule comprising a nucleotide sequence encoding a tubulin molecule, wherein said nucleic acid molecule comprises the sequence according to SEQ. ID. No. 1

This problem is solved in a second aspect by a nucleic acid molecule comprising a nucleotide sequence encoding a tubulin molecule, wherein said nucleic acid molecule comprises the sequence according to SEQ. ID. No. 2.

This problem is solved in a third aspect by a nucleic acid molecule comprising a nucleotide sequence encoding the C terminal end of a tubulin molecule, wherein the C terminal end comprises the following sequence:

VLEEDEEVTEEAEMEPEDKGH (SEQ ID. No. 4)

This problem is solved in a fourth aspect by a nucleic acid molecule comprising a nucleotide sequence encoding a tubulin molecule, wherein said nucleic acid molecule hybridises to the nucleic acid molecule selected from the group comprising SEQ. ID. No. 1, SEQ. ID. No 2, SEQ. ID. No. 4 and derivatives therefrom.

This problem is solved in a fifth aspect by a nucleic acid molecule comprising a nucleotide sequence encoding a tubulin molecule, wherein the tubulin molecule or part thereof comprises an amino acid sequence according to SEQ. ID. No. 3.

This problem is solved in a sixth aspect by a nucleic acid molecule comprising a nucleotide sequence encoding a tubulin molecule, wherein the nucleotide nucleic acid molecule would hybridise, but for the degeneracy of the genetic code, to any of the nucleic acid molecule and/or nucleotide sequence according to the above aspects of the invention.

In a further aspect the problem is solved by an amino acid sequence of a tubulin molecule, preferably of a beta-tubulin molecule and more preferably of a class VI beta-tubulin, wherein the amino acid sequence is coded by a nucleic acid molecule according to the present invention or part thereof.

In a preferred embodiment of the various aspects of the invention the tubulin molecule is the tubulin molecule expressed in haematopoietic tissue, in an even more preferred embodiment the tubulin molecule is preferably expressed in haematopoietic tissue.

In a further preferred embodiment of the various aspects of the invention the tubulin molecule is β-tubulin, preferably an isotype class VI beta-tubulin, most preferably a human homologue of the vertebrate beta tubulin isotype class VI.

In a still further preferred embodiment of the various aspects of the invention the nucleic acid molecule is an isolated nucleic acid molecule.

In a seventh aspect of the invention the problem is solved by a vector comprising any of the nucleic acid molecules of the present invention.

In a preferred embodiment the vector is an expression vector.

In a eighth aspect of the invention the problem is solved by a cell, preferably a host cell, comprising the vector according to the invention.

In a nineth aspect of the invention the problem is solved by a method for the production of a tubulin molecule, preferably of a beta- tubulin molecule and more preferably of a class VI beta-tubulin, characterized by culturing the cell according to the present invention and isolating the protein product.

In a tenth aspect of the invention the problem is solved by a method for diagnosing polymorphism in a tubulin molecule or gene, preferably β-tubulin, and more preferably an isotype class VI beta-tubulin, most preferably a human homologue of the vertebrate beta tubulin isotype class VI or in the nucleic acid coding such tubulin, wherein a nucleic acid according to the present invention is used.

In a eleventh aspect of the invention the problem is solved by the use of a nucleic acid according to the present invention for diagnosis.

In a twelfth aspect of the invention the problem is solved by a method for the screening for anticancer drugs using a nucleic acid according to any of claims 1 to 9 or any transcription and/or translation product thereof.

In a further aspect of the invention the problem is solved by the use of a nucleic acid according to the present invention for screening purposes.

The inventors of the present invention have surprisingly found the human homologue of the vertebrate beta-tubulin isotype class VI. This homologue can be characterised by the C-terminal amino acid sequence. A comparison of the C-terminal isotype defining sequences including the one of the human homologue referred to herein also as TUBB1, can be taken from table 1.

TABLE 1
C-terminal isotype defining sequences.

| | C-terminal | |
|---|---|---|
| Isotype | isotype defining sequence | gene/protein |
| Class I | EEEEDFGEEAEEEA | cb7 |
| Class II | DEQGEFEEEGEEDEA | cb1/cb2 |
| | EG | mb2 |
| Class III | EEEGEMYEDDEEESEQGAK | cb4 |
| | P | hb4 |
| Class IVa | EEGEFEEEAEEEVA | mb4 |
| Class IVb | EEEGEFEEEAEEEAE | cb3 |
| | VA | mb3 |
| Class V | NDGEEEAFEDDEEEINE | cb5 |
| Class VI | DVEEYEEAEASPEKET | cb6 |
| | GLEDSEEDAEEAEVEAEDKDH | mb1 |
| | V ED VT M P G | TUBB1 |

The remainder of the sequences are identical. Particularly focussing on the C-terminal isotype defining sequences it is to be noted that the class VI isotypes are so variable that the sequences of chicken (cb6) and mouse (mb1) are completely specified whereas TUBB1 is rather homologue so that the only differences of TUBB1 relative to mb1 are indicated. In connection with the beta-tubulin isotypes of table 1 <<h>> refers to human, <<c>> to chicken and <<m>>to mouse>>. The h homologue TUBB1 shows a homology of 92% relative to mb1 on the basis of the 451 AS translated out of the potential ORF.

Derivatives of the various nucleic acids described herein are particularly those nucleic acids which encode the same amino acids as does the non-derivative nucleic acid, or those nucleic acids which show the same chemical, biological or structural characteristics as does the non-derivative one. Such derivative typically comprises at least some nucleobases which differ from the nucleobases of the non-derivative nuleic acids. Preferably a pyrimidine nucleobase is replaced by another pyrimidine nucleobase and a purine nucleobase replaced by a different purine nucleobase. Derivatives of a nucleic acid in the meaning of this invention may also be such nucleic acid (and amino acids possibly derived therefrom) which hybridise to said nucleic acid. More particularly, such hybridisation is carried out using an oligonucleotide comprising the following sequence (SEQ ID No. 14):

GTT CTA GAG GAA GAT GAA GAG GTC ACG GAG GAG GCA GAA
ATG GAG CCA GAA GAT AAG GGA CAT

As probe for stringent hybridisation meaning a hybridisation solution with a Na+ concentration of 0.4 M and a hybridisation temperature range of 55 to 60° C. SEQ. ID. No. 14 is based on SEQ ID. No. 4

The C-terminal sequence of TUBB1 is the part of the beta-tubulin where MAPs (microtubuli associated proteins) preferably bind which are proteins partially unknown having an impact on association of microtubuli. Similar to the mouse homologue mb1 (Wang et. al 1986, J. Cell Biol. 103: 1903-1910) and its translation product (Kavallaris et al. 1997, J. Clin. Invest. 100: 1282-1293) the TUBB1 protein is found in the cells of the hematopoietic tissue as shown for mb1 in bone marrow and thrombocytes by Western blot analysis. It is understood that the protein of isotype VI is specific for the Marginal Band in nucleated fetal erythroblasts and platelets, respectively (Lewis et al. 1987, Cell 49 (4): 539-48)

Having the sequence of the human homologue of beta-tubulin isotype class VI now at hand, various applications thereof are possible. One such application is the screening for cytostatic drugs. Basically, one such approach is the same as described by Kavallaris (supra) the disclosure of which is herein incorporated by reference, and is based on the interference of a candidate cytostatic drug with the aggregation of the tubulin molecules. Such screening can be carried out in cell culture.

A further application is the use of the TUBB1 and its amino acid and nucleic acid sequence, respectively, for the diagnosis of polymorphic regions within the TUBB1 gene and for a predisposition for distinct diseases such as leucaemia and other diseases related to haematopoiesis.

Vectors useful in the practice of the present invention are known to the one skilled in the art, particularly those which provide for a further method for purification of the expressed protein such as pPROTet.E/pPROLar.A (Clontech). With regard to a possible posttranslational processing an expression in a baculovirus system is also envisaged which means that vectors such as, e.g., pBacPAK 8/9 (Clontech) may be used. Inducible vector systems may also be used, preferably for testing the effect of TUBB1 in a cellular environment. Such testing may be part of a test for therapeutical approaches. Representative vectors and cellular systems are pRevTRE (Clontech) and HeLa (epithelial carcinoma, cervix). Other suitable cell lines useful in the practice of the present invention are known to the one skilled in the art.

Further molecular biological methods useful in the practice of the present invention are, e.g., described in Maniatis et al. (Maniatis T., Fritsch E. F., Sambrook J., (1982) Cold Spring Harbor Press, Cold Spring Harbor).

The invention is now further characterized by reference to the sequences of the sequence protocol, the figures and the examples from which further embodiments, features and advantages can be taken, wherein

SEQ. ID. No. 1 is the cDNA sequence of TUBB1;

SEQ. ID. No. 2 is the open reading frame of the cDNA;

SEQ. ID. No.3 is the amino acid sequence of the open reading frame of SEQ. ID. No. 2;

SEQ. ID. No. 4 is the C-terminal isotype identifying sequence of the human homologue of the beta-tubulin isotype class VI;

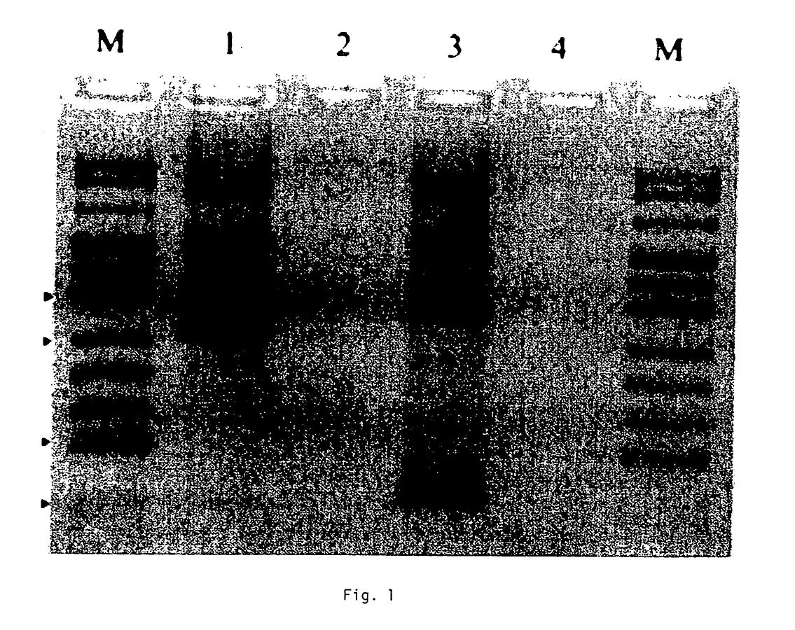

FIG. 1 shows a 5′RACE for H57679 using primers AP2/H57E3F (1) and AP2/H57E1F (3);

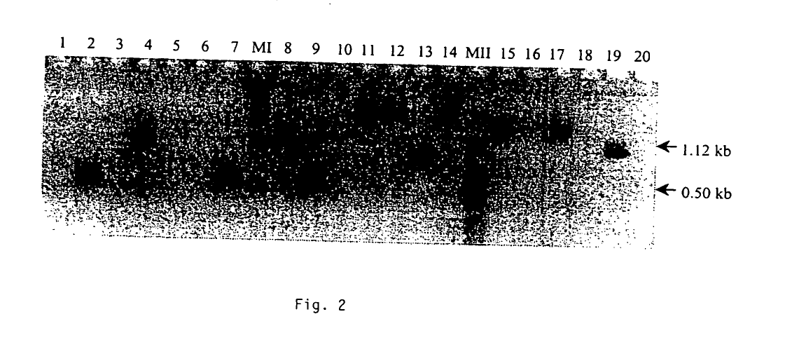

FIG. 2 shows subclones of J0464 for the sequencing; and

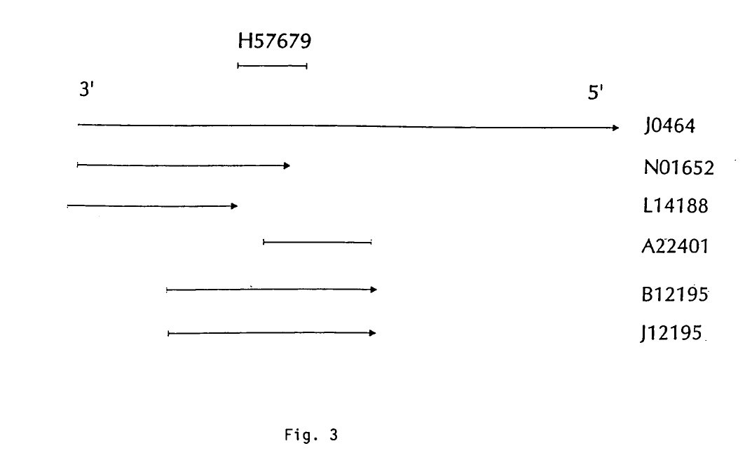

FIG. 3 shows the arrangement of the cDNA clones for H57679.

FIG. 1 shows the 5′RACE for H57679 out of a marathon-ready-cDNA library. Lane 1 shows a 260 bp fragment amplified during PCR using sequence specific primer H57E2F and adaptor specific primer AP2 whereas lane 2 shows similar fragments using different sequence specific primer H57E1F and AP2. Lanes 3 and 4 are control PCR to exclude any contamination.

Sequencing of the 3.3 kb insert of J0464 is based on producing several subclones using the Genome Priming System (NEB, New England Biolabs, Schwalbach, Germany). The statistical integration of a Transposon element allows a sequence specific selection of positive clones using PCR as shown in FIG. 2. PCR analysis of 20 subclones using sequence specific primer H57679L and the Transposon specific primer Tn7R shows several different integration sides which could be used for sequencing of overlapping fragments.

| | | |
|---|---|---|
| H57679L: | tgactggcatcctgctttc | (SEQ. ID. No. 5) |
| Tn7R: | gcataactggactgatttc | (SEQ ID. No. 6) |

### EXAMPLE 1

### Characterisation of TUBB1

The gene of SEQ. ID. No. 1, referred to herein as TUBB1, was characterized starting from an EST in the distal part of chromosome 20 q which was taken from the Radiation Hybrid Map (RH4) which is a database based physical map of the human genome. The Radiation Hybrid Database by Patricia Rodriguez-To\acute{m}, Carsten Helgesen and Philip Lijnzaad can be characterised as follows: The EBI (EMBL Outstation, The European Bioinformatics Institute, Wellcome Trust Genome Campus, Hinxton, Cambridge CB10 1SD,UK; since July 1995) maintains RHdb, a public database for radiation hybrid mapping, including raw results and maps. RH data are used in the generation of alternative genetic maps as they can include non-polymorphic markers. The main type of entry in the database consists of PCR hybridization results of a particular STS on a particular panel. STS belonging to one (or more) of the following categories are used for mapping:

Genetic markers

Expressed Sequences Tags (EST)

Fully sequenced cDNA

CpG Islands

Random genomic human clones

Chromosome specific libraries.

Cross-references to a number of related databases are also stored. These include Genbank/EMBL, dbEST, GDB, and the databases of the laboratories that did the experiments. The database is maintained in the Oracle Relational Database Management System (RDBMS).

The Radiation Hybrid Map itself was taken from the website of NCBI (National Center for Biotechnology Information (NCBI); National Center for Biotechnology Information, National Library of Medicine, Building 38A, Room 8N805, Bethesda, Md. 20894). The particular EST chosen is H 57679. EST 57679 is one out of many ESTs located in the distal area of chromosome 20. The entry of the EMBL database reads as follows:

LOCUS H57679 433 bp mRNA EST 05, Oct. 1995

DEFINITION yr27b11.s1 Soares fetal liver spleen 1NFLS Homo sapiens cDNA clone

IMAGE:206493 3′, MRNA sequence.

ACCESSION H57679

VERSION H57679.1 GI:1010511

KEYWORDS EST.

SOURCE human.

ORGANISM Homo sapiens Eukaryota; Metazoa; Chordata; Craniata; Vertebrata; Euteleostomi; Mammalia; Eutheria; Primates; Catarrhini; Hominidae; Homo.

REFERENCE 1 (bases 1 to 433)

AUTHORS Hillier, L., Clark, N., Dubuque, T., Elliston, K., Hawkins, M., Holman, M., Hultman, M., Kucaba,T., Le, M., Lennon, G., Marra, M., Parsons, J., Rifkin, L., Rohlfing, T., Soares, M., Tan, F., Trevaskis, E., Waterston, R., Williamson, A., Wohldmann, P. and Wilson, R.

TITLE The WashU-Merck EST Project

JOURNAL Unpublished

COMMENT Contact: Wilson RK Washington University School of Medicine, 4444 Forest Park Parkway, Box 8501, St. Louis, Mo. 63108.

The cDNA clone corresponding to EST H 57679 was ordered from the RZPD in Berlin, Germany (Resource Center/Primary Database: Heubnerweg 6, 14059 Berlin)

After sequencing of the cDNA clone using standard primers (M13rev, -21M13for, T3, T7), respectively, having the following sequences:

| | |
|---|---|
| −21M13forward: | |
| TGTAAAACGACGGCCAGT | (SEQ. ID. No. 7) |
| and | |
| M13reverse: | |
| CAGGAAACAGCTATGACC, | (SEQ. ID. No. 8) |
| T3: | |
| AATTAACCCTCACTAAAGGG; | (SEQ. ID. No. 9) |
| T7: | |
| TAATACGACTCACTATAGGG, | (SEQ. ID. NO. 10) |

primers were derived therefrom and used for the screening of further cDNA clones from tissue specific cDNA libraries of the RZPD (multiple tissue pools; MTP) which are also referred to as “GeneFinder” pools.

The following further clones were found:

| name of the clone | seize of the insert |
|---|---|
| ICRFp512B12195Q1 | 1.7 kb |
| ICRFp5I2J0464Q1 | 3.3 kb |
| ICRFp512J12195Q1 | 1.7 kb |
| ICRFp512L14188Q1 | 1.3 kb |

For further verification of the completeness of the 5′end of the cDNA a RACE-PCR from Marathon-Ready-cDNA (CLONTECH GmbH, Tullastr. 4, 69126 Heidelberg, Germany) was run using the following primers:

| | | |
|---|---|---|
| AP2 | actcactatagggctcgagcggc | (SEQ. ID. No. 11) |
| H57E1F | cacggaatcctcagactctcag | (SEQ. ID. No. 12) |
| H57E2F | gttgtagtacacgctgattctctcc | (SEQ. ID. No. 13) |

AP2 is an adaptor specific primer. The primers H57 E1F and H57E2F are specific for sequences of the 5′part of clone ICRFp512J0464Q1.

The details of the RACE-PCR can be summarized as follows: In a first round an adaptor specific and a gene specific primer are used; in the second round a re-amplification is carried our using the same primers.

1st round:

| | | |
|---|---|---|
| 10 | pg/μl | Marathon-ready-cDNA |
| 1x | | cDNA PCR-reaction buffer (Clontech) |
| 0.2 | mM | dNTPs |
| 1x | | Advantage cDNA Pol. mix |
| 0.2 | pmol | Adaptor primer |
| 0.2 | pmol | H57E2F |
| ad 5 | μl H₂O | |
| 1:50 | | out of 1. |
| 1x | | Elongase buffer (Gibco BRL) |
| 0.5 | mM | dNTPs |
| 1.95 | mM | MgCl₂ |
| 0.25 | μl | Elongase (Gibco BRL) |
| 0.2 | pmol | Adaptor primer |
| 0.2 | pmol | H57E2F |
| ad 10 | μl H₂O | |

PCR:

| | |
|---|---|
| 22x | 92° C. - 50 s |
| | 72° C. - 50 s |
| | (−1° C. per cycle) |
| | 68° C. - 4 min |
| 25x | 92° C. - 50 s |
| | 60° C. - 50 s |
| | 68° C. - 4 min |

A PCR product of about 260 bp was amplified and directly sequenced by using the primers H57E2F/AP2 applied in the RACE-PCR.

Analysis of the results indicated that the 5′ end of ICRFp512J0464Q1 was to be regarded as complete as the sequence of the Marathon product was identical except four bases which confirmed the sequence of the clone. As the 3 ′ends of all clones were nearly identical there is now a complete or full-length cDNA for this locus/gene on chromosome 20q.

### EXAMPLE 2

Sequences homologous to TUBB1

With regard to the fact that typically isotypes of one class of tubulins share a high degree of homology between various species a respective analysis of class VI isotype beta-tubulins was performed on homology at the amino acid level the results of which are summarized in the following table. The basis for this analysis is the amino acid sequence of TUBB1 corresponding to SEQ. ID. No. 3.

Table

| | Acc. Nr. | | | | |
|---|---|---|---|---|---|
| | Ident. | Id. Pos. | Pos. | (%) | (%) |
| Mouse beta tubulin mb1 | | 413/451 | 438/451 | 92 | 97 |
| Chicken class VI | P09207 | 353/430 | 389/430 | 82 | 90 |
| beta tubulin | | | | | |

Ident. = identical amino acid
Pos. = identical plus amino acid of the same functional group

Both mouse beta tubulin mb1 and chicken class VI beta tubulin are described in literature. Chicken: Murphy D. B., Wallis K. T., Machlin P. S., Ratrie H. III, Cleveland D. W.; “The sequence and expression of the divergent β-tubulin in chicken erythrocytes.”; J. Biol. Chem. 262:14305-14312(1987)). The accession number refers to SWISSPROT; the cDNA is also available from EMBL under accession number J02828.

The mouse sequence is available neither from EMBL nor from SWISSPROT, but was published by Wang et al. (Wang D, Villasante A, Lewis S A, Cowan N J “The mammalian beta-tubulin repertoire: hematopoietic expression of a novel, heterologous beta-tubulin isotype” J. Cell Biol. 103(5):1903-10 (1986)).

The features disclosed in the specification, the figures, sequence protocol and the claims may be, individually or in combination, essential for the realization of the invention in its various embodiments.

1. A nucleic acid molecule comprising a nucleotide sequence encoding a tubulin molecule, wherein said nucleic acid molecule comprises the sequence according to SEQ. ID. No. 1

2. A nucleic acid molecule comprising a nucleotide sequence encoding a tubulin molecule, wherein said nucleic acid molecule comprises the sequence according to SEQ. ID. No. 2.

3. A nucleic acid molecule comprising a nucleotide sequence encoding the C terminal end of a tubulin molecule, wherein the C terminal end comprises the following sequence:
VLEEDEEVTEEAEMEPEDKGH (SEQ ID. No. 4)

4. A nucleic acid molecule comprising a nucleotide sequence encoding a tubulin molecule, wherein said nucleic acid molecule hybridizes to the nucleic acid molecule selected from the group comprising SEQ. ID. ID. No. 1, SEQ. ID. No 2, SEQ. ID. No. 4 and derivatives therefrom.

5. A nucleic acid molecule comprising a nucleotide sequence encoding a tubulin molecule, wherein the tubulin molecule or part thereof comprises an amino acid sequence according to SEQ. ID. No. 3.

6. A nucleic acid molecule comprising a nucleotide sequence encoding a tubulin molecule, wherein the nucleotide nucleic acid molecule would hybridize, but for the degeneracy of the genetic code, to the nucleic acid molecule and/or the nucleotide sequence according to any of claims 1 to 5.

7. The nucleic acid molecule according to any of claims 1 to 6, wherein the tubulin molecule is the tubulin molecule expressed in haematopoietic tissue.

8. The nucleic acid molecule according to any of claims 1 to 7, wherein the tubulin molecule is β-tubulin, preferably an isotype class VI beta-tubulin, most preferably a human homologue of the vertebrate beta tubulin isotype class VI.

9. The nucleic acid molecule according to any of claims 1 to 8, wherein the nucleic acid molecule is an isolated nucleic acid molecule.

10. A vector comprising the nucleic acid molecule according to any of claims 1 to 9.

11. The vector according to claim 10, wherein the vector is an expression vector.

12. A cell, preferably a host cell, comprising the vector according to any of claims 10 or 11.

13. An amino acid sequence of a tubulin molecule, preferably of a beta-tubulin molecule and more preferably of a class VI beta-tubulin, wherein the amino acid sequence is encoded by a nucleic acid molecule according to any of claims 1 to 9 or part thereof.

14. A method for the production of a tubulin molecule, preferably of a beta-tubulin molecule and more preferably of a class VI beta-tubulin, characterized by culturing the cell according to claim 12 and isolating the protein product.

15. A method for diagnosing polymorphism in a tubulin molecule, preferably β-tubulin, and more preferably an isotype class VI beta-tubulin, most preferably a human homologue of the vertebrate beta tubulin isotype class VI or in the nucleic acid coding such tubulin, wherein a nucleic acid according to any of claims 1 to 9 is used.

16. Use of a nucleic acid according to any of claims 1 to 9 for diagnosis.

17. A method for the screening of anticancer drugs using a nucleic acid according to any of claims 1 to 9 or any transcription and/or translation product thereof.

18. Use of a nucleic acid according to any of claims 1 to 9 for screening purposes.

19. Use of a nucleic acid according to any of claims 1 to 9 as a medicament.
